SEQUENCE LISTING (16 sequences)

SEQ ID NO: 1 - protein, 330 aa, Artificial Sequence
  Description of Artificial Sequence Synthetic polypeptide
MAFSADTPEIVYTHDTGLDYITYSDYELDPANPLAGGAAWIEGAFVPPSEARISIFDQGFYTSDATYTVFHVWNGNAFRLGDHIERLFSNAESIRLIPPLTQDEVKEIALELVAKTELREAIVWVAITRGYSSTPLERDVTKHRPQVYMYAVPYQWIVPFDRIRDGVHLMVAQSVRRTPRSSIDPQVKNFAAGDLIRAIQETHDRGFELPLLLDFDNLLAEGPGFNVVVIKDGVVRSPGRAALPGITRKTVLEIAESLGHEAILADITPAELRDADEVLGCSTAGGVWPFVSVDGNSISDGVPGPVTQSIIRRYWELNVEPSCLLTPVQY

SEQ ID NO: 2 - protein, 330 aa, Artificial Sequence
  Description of Artificial Sequence Synthetic polypeptide
MAFSLDTPEIVYTHDTGLDYITYSDYELDPANPLAGGAAWIEGAFVPPSEARISIFDQGFYTSDATYTVFHVWNGNAFRLGDHIERLFSNAESIRLIPPLTQDEVKEIALELVAKTELREAIVWVAITRGYSSTPLERDVTKHRPQVYMYAVPYQWIVPFDRIRDGVHLMVAQSVRRTPRSSIDPQVKNFAAGDLIRAIQETHDRGFELPLLLDFDNLLAEGPGFNVVVIKDGVVRSPGRAALPGITRKTVLEIAESLGHEAILADITPAELRDADEVLGCSTAGGVWPFVSVDGNSISDGVPGPVTQSIIRRYWELNVEPSCLLTPVQY

SEQ ID NO: 3 - protein, 330 aa, Artificial Sequence
  Description of Artificial Sequence Synthetic polypeptide
MAFSLDTPEIVYTHDTGLDYITYSDYELDPANPLAGGAAWIEGAFVPPSEARISVFDQGFYTSDATYTVFHVWNGNAFRLGDHIERLFSNAESIRLIPPLTQDEVKEIALELVAKTELREAMVWVAITRGYSSTPLERDVTKHRPQVYMYAVPYQWIVPFDRIRDGVHLMVAQSVRRTPRSSIDPQVKNFAAGDLIRAIQETHDRGFELPLLLDHDNLLAEGPGFNVVVIKDGVVRSPGRAALPGITRKTVLEIARSLGHEAILADITPAELRDADEVLGCSTAGGVWPFVSVDGNSISDGVPGPVTQSIIRRYWELNVEPSCLLTPVQY

SEQ ID NO: 4 - protein, 330 aa, Artificial Sequence
  Description of Artificial Sequence Synthetic polypeptide
MAFSLDTPEIVYTHDTGLDYITYSDYELDPANPLAGGAAWIEGAFVPPSEARISVFDQGFYTSDATYTVFHVWNGNAFRLGDHIERLFSNAESIRLIPPLTQDEVKEIALELVAKTELREAMVWVAITRGYSSTPLERDVTKHRPQVYMYAVPYQWIVPFDRIRDGVHLMVAQSVRRTPRSSIDPQVKNFASIDLIRAIQETHDRGFELPLLLDHDNLLAEGPGFNVVVIKDGVVRSPGRAALPGITRKTVLEIAESLGHEAMLADITPAELRDADEVLGCSTAGGVWPFVSVDGNSISDGVPGPVTQSIIRRYWELNVEPSCLLTPVQY

SEQ ID NO: 5 - protein, 330 aa, Artificial Sequence
  Description of Artificial Sequence Synthetic polypeptide
MAFSLDTPEIVYTHDTGLDYITYSDYELDPANPLAGGAAWIEGAFVPPSEARISVFDQGFYTSDATYTAFHVWNGNAFRLGDHIERLFSNAESIRLIPPLTQDEVKEIALELVAKTELREAMVWVAITRGYSSTPLERDVTKHRPQVYMYAVPYQWIVPFDRIRDGVHLMVAQSVRRTPRSSIDPQVKNFASIDLIRAIQETHDRGFELPLLLDHDNLLAEGPGFNVVVIKDGVVRSPGRAALPGITRKTVLEIAESLGHEAMLADITPAELRDADEVLGCSTAGGVWPFVSVDGNSISDGVPGPVTQSIIRRYWELNVEPSCLLTPVQY

SEQ ID NO: 6 - protein, 330 aa, Artificial Sequence
  Description of Artificial Sequence Synthetic polypeptide
MAFSLDTPEIVYTHDTGLDYITYSDYELDPANPLAGGAAWIEGAFVPVSEARISVFDQGFYASDATYTAFHVWNGNAFRLGDHIERLWSNAESIRLIPPLTQDEVKEIALELVAKTELREAMVGVAITRGYSSTPLERDVTKHRPQVYMYAVPYQWIVPFDRIRDGVHLMVAQSVRRTPRSSIDPQVKNFASIDLIRAIQETHDRGFELPLLLDHDNLLAEGPGFNVVVIKDGVVRSPGRAALPGITRKTVLEIARSLGHEAMLADITPAELRDADEVLGCSTAGGVWPFVSVDGNSISDGVPGPVTQSIIRRYWELNVEPSCLLTPVQY

SEQ ID NO: 7 - protein, 330 aa, Artificial Sequence
  Description of Artificial Sequence Synthetic polypeptide
MAFSLDTPEIVYTHDTGLDYITYSDYELDPANPLAGGAAWIEGAFVPVSEARISVFDQGFYASDATYTAFHVWNGNAFRLGDHIERLWSNAESIRLIPPLTQDEVKEIALELVAKTELREAMVGVVITRGYSSTPLERDVTKHRPQVYMYAIPYQWIVPFDRIRDGVHLMVAQSVRRTPRSSIDPQVKNFASIDLIRAIQETHDRGFELPLLLDHDNLLAEGPGFNVVVIKDGVVRSPGRAALPGITRKTVLEIARSLGHEAMLADITPAELRDADEVLGCSTAGGVWPFVSVDGNSISDGVPGPVTQSIIRRYWELNVEPSCLLTPVQY

SEQ ID NO: 8 - protein, 330 aa, Artificial Sequence
  Description of Artificial Sequence Synthetic polypeptide
MAFSLDTPEIVYTHDTGLDYITYSDYELDPANPLAGGAAWIEGAFVPPSEARISVFDQGFYTSDATYTAFHVWNGNAFRLGDHIERLFSNAESIRLIPPLTQDEVKEIALELVAKTELREAMVWVAITRGYSSTPLERDVTKHRPQVYMYAVPYQWIVPFDRIRDGVHLMVAQSVRRTPRSSIDPQVKNFAAGDLIRAIQETHDRGFELPLLLDHDNLLAEGPGFNVVVIKDGVVRSPGRAALPGITRKTVLEIARSLGHEAILADITPAELRDADEVLGCSTAGGVWPFVSVDGNSISDGVPGPVTQSIIRRYWELNVEPSCLLTPVQY

SEQ ID NO: 9 - DNA, 993 bp, Artificial Sequence
  Description of Artificial Sequence Synthetic polynucleotide
atggcgttctcagcggacacccctgaaatcgtttacacccacgacaccggtctggactatatcacctactctgactacgaactggacccggctaacccgctggctggtggtgccgcttggatcgaaggtgctttcgttccgccgtctgaagctcgtatctctatcttcgaccagggtttttatacttctgacgctacctacaccgtcttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaattgcgtgaagcgattgtttgggttgcaatcacccgtggttactcttctaccccattggagcgtgacgtcaccaaacatcgtccgcaggtttacatgtatgctgttccgtaccagtggatcgtaccgtttgaccgcatccgtgacggtgttcacctgatggttgctcagtcagttcgtcgtacaccgcgtagctctatcgacccgcaggttaaaaacttcgcggcaggtgacctgatccgtgcaattcaggaaacccacgaccgtggtttcgagttaccgctgctgctggactttgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggctgacatcaccccggctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtactaa

SEQ ID NO: 10 - DNA, 993 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccgccgtctgaagctcgtatctctatcttcgaccagggtttttatacttctgacgctacctacaccgtcttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaattgcgtgaagcgattgtttgggttgcaatcacccgtggttactcttctaccccattggagcgtgacgtcaccaaacatcgtccgcaggtttacatgtatgctgttccgtaccagtggatcgtaccgtttgaccgcatccgtgacggtgttcacctgatggttgctcagtcagttcgtcgtacaccgcgtagctctatcgacccgcaggttaaaaacttcgcggcaggtgacctgatccgtgcaattcaggaaacccacgaccgtggtttcgagttaccgctgctgctggactttgacaacctgctggctgaaggtccgggtttcaacgttgttgttatcaaagacggtgttgttcgttctccgggtcgtgctgctctgccgggtatcacccgtaaaaccgttctggaaatcgctgaatctctgggtcacgaagctatcctggctgacatcaccccggctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtactaa

SEQ ID NO: 11 - DNA, 990 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccgccgtctgaagctcgtatctctgttttcgaccagggtttttatacttctgacgctacctacaccgtcttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaattgcgtgaagcgatggtttgggttgcaatcacccgtggttactcttctaccccattggagcgtgacgtcaccaaacatcgtccgcaggtttacatgtatgctgttccgtaccagtggatcgtaccgtttgaccgcatccgtgacggtgttcacctgatggttgctcagtcagttcgtcgtacaccgcgtagctctatcgacccgcaggttaaaaacttcgcggcgggcgacctgatccgtgcaattcaggaaacccacgaccgtggtttcgagttaccgctgctgctggaccacgacaacctgctggctgaaggtccgggtttcaacgttgttgttatcaaagacggtgttgttcgttctccgggtcgtgctgctctgccgggtatcacccgtaaaaccgttctggaaatcgctcgttctctgggtcacgaagctattctggctgacatcaccccggctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtac

SEQ ID NO: 12 - DNA, 990 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccgccgtctgaagctcgtatctctgttttcgaccagggtttttatacttctgacgctacctacaccgtcttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaattgcgtgaagcgatggtttgggttgcaatcacccgtggttactcttctaccccattggagcgtgacgtcaccaaacatcgtccgcaggtttacatgtatgctgtgccgtaccagtggatcgtaccgtttgaccgcatccgtgacggtgttcacctgatggttgctcagtcagttcgtcgtacaccgcgtagctctatcgacccgcaggttaaaaacttcgcgtcgattgacctgatccgtgcaattcaggaaacccacgaccgtggtttcgagttaccgctgctgctggaccacgacaacctgctggctgaaggtccgggtttcaacgttgttgttatcaaagacggtgttgttcgttctccgggtcgtgctgctctgccgggtatcacccgtaaaaccgttctggaaatcgctgagtctctgggtcacgaagctatgctggctgacatcaccccggctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtac

SEQ ID NO: 13 - DNA, 993 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccgccgtctgaagctcgtatctctgttttcgaccagggtttttatacttctgacgctacctacaccgctttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaattgcgtgaagcgatggtttgggttgcaatcacccgtggttactcttctaccccattggagcgtgacgtcaccaaacatcgtccgcaggtttacatgtatgctgtgccgtaccagtggatcgtaccgtttgaccgcatccgtgacggtgttcacctgatggttgctcagtcagttcgtcgtacaccgcgtagctctatcgacccgcaggttaaaaacttcgcgtcgattgacctgatccgtgcaattcaggaaacccacgaccgtggtttcgagttaccgctgctgctggaccacgacaacctgctggctgaaggtccgggtttcaacgttgttgttatcaaagacggtgttgttcgttctccgggtcgtgctgctctgccgggtatcacccgtaaaaccgttctggaaatcgctgagtctctgggtcacgaagctatgctggctgacatcaccccggctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtactaa

SEQ ID NO: 14 - DNA, 993 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccggtctctgaagctcgtatctctgttttcgaccagggtttttatgcctctgacgctacctacaccgctttccacgtttggaacggtaacgctttccgtctgggggaccacatcgaacgtctgtggtctaatgcggaatctattcgtttgatcccgccgctgacccaggacgaagttaaagagatcgctctggaactggttgctaaaaccgaattgcgtgaagcgatggttggggttgcaatcacccgtggttactcttctaccccattggagcgtgacgtcaccaaacatcgtccgcaggtttacatgtatgctgtcccgtaccagtggatcgtaccgtttgaccgcatccgtgacggtgttcacctgatggttgctcagtcagttcgtcgtacaccgcgtagctctatcgacccgcaggttaaaaacttcgcgtcgattgacctgatccgtgcaattcaggaaacccacgaccgtggtttcgagttaccgctgctgctggaccacgacaacctgctggctgaaggtccgggtttcaacgttgttgttatcaaagacggtgttgttcgttctccgggtcgtgctgctctgccgggtatcacccgtaaaaccgttctggaaatcgctcgttctctgggtcatgaagctatgctggctgacatcaccccagctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtactaa

SEQ ID NO: 15 - DNA, 993 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccggtctctgaagctcgtatctctgttttcgaccagggtttttatgcctctgacgctacctacaccgctttccacgtttggaacggtaacgctttccgtctgggggaccacatcgaacgtctgtggtctaatgcggaatctattcgtttgatcccgccgctgacccaggacgaagttaaagagatcgctctggaactggttgctaaaaccgaattgcgtgaagcgatggttggggttgtaatcacccgtggttactcttctaccccattggagcgtgacgtcaccaaacatcgtccgcaggtttacatgtatgctattccgtaccagtggatcgtaccgtttgaccgcatccgtgacggtgttcacctgatggttgctcagtcagttcgtcgtacaccgcgtagctctatcgacccgcaggttaaaaacttcgcgtcgattgacctgatccgtgcaattcaggaaacccacgaccgtggtttcgagttaccgctgctgctggaccacgacaacctgctggctgaaggtccgggtttcaacgttgttgttatcaaagacggtgttgttcgttctccgggtcgtgctgctctgccgggtatcacccgtaaaaccgttctggaaatcgctcgttctctgggtcatgaagctatgctggctgacatcaccccagctgaactgcgtgatgccgacgaagttctgggttgctcaaccgcgggtggtgtttggccgttcgtttctgttgacggtaactcgatctctgacggtgttccgggtccggttacccagtctatcatccgtcgttactgggaactgaacgttgaaccttcttgcctgctgaccccggtacagtactaa

SEQ ID NO: 16 - DNA, 993 bp, Artificial Sequence
  Description of Artificial Sequence Synthetic polynucleotide
atggcgttctcactggacacccctgaaatcgtttacacccacgacaccggtctggactatatcacctactctgactacgaactggacccggctaacccgctggctggtggtgccgcttggatcgaaggtgctttcgttccgccgtctgaagctcgtatctctgttttcgaccagggtttttatacttctgacgctacctacaccgctttccacgtttggaacggtaacgctttccgtctgggggaccacatcgaacgtctgttctctaatgcggaatctattcgtttgatcccgccgctgacccaggacgaagttaaagagatcgctctggaactggttgctaaaaccgaattgcgtgaagcgatggtttgggttgcaatcacccgtggttactcttctaccccattggagcgtgacgtcaccaaacatcgtccgcaggtttacatgtatgctgttccgtaccagtggatcgtaccgtttgaccgcatccgtgacggtgttcacctgatggttgctcagtcagttcgtcgtacaccgcgtagctctatcgacccgcaggttaaaaacttcgcggcgggcgacctgatccgtgcaattcaggaaacccacgaccgtggtttcgagttaccgctgctgctggaccacgacaacctgctggctgaaggtccgggtttcaacgttgttgttatcaaagacggtgttgttcgttctccgggtcgtgctgctctgccgggtatcacccgtaaaaccgttctggaaatcgctcgttctctgggtcacgaagctattctggctgacatcaccccggctgaactgcgtgatgccgacgaagttctgggttgctcaaccgcgggtggtgtttggccgttcgtttctgttgacggtaactctatctctgacggtgttccgggtccggttacccagtctatcatccgtcgttactgggaactgaacgttgaaccttcttgcctgctgaccccggtacagtactaa

(19) United States
(12) Patent Application Publication
(10) Pub. No.: US 2024/0247240 A1
(43) Pub. Date: Jul. 25, 2024

(54) TRANSAMINASE ENZYMES FOR THE TRANSAMINATION OF (1S,5R)-6,8-DIOXABICYCLO[3.2.1]OCTAN-4-ONE

(71) Applicant: MERCK SHARP & DOHME LLC, Rahway, NJ (US)

(72) Inventors: Karla M. Camacho Soto, Rahway, NJ (US); Wai Ling Cheung-Lee, Rahway, NJ (US); Hsing-I Ho, New Providence, NJ (US); John McIntosh, Metuchen, NJ (US); Grant S. Murphy, Princeton, NJ (US); Weilan Pan, Downingtown, PA (US); Christopher K. Prier, Newark, NJ (US); Deeptak Verma, Feasterville-Trevose, PA (US)

(73) Assignee: MERCK SHARP & DOHME LLC, Rahway, NJ (US)

(21) Appl. No.: 18/563,157

(22) PCT Filed: May 26, 2022
(86) PCT No.: PCT/US2022/030992

§ 371 Date: Nov. 21, 2023

Related U.S. Application Data
(60) Provisional application No. 63/339,210, filed on May 6, 2022.
(60) Provisional application No. 63/194,298, filed on May 28, 2021.

(51) Int. Cl. C12N 9/10; C12N 15/70; C12P 17/18

(52) U.S. Cl. CPC C12N 9/1096; C12N 15/70; C12P 17/181; C12Y 206/01

(57) ABSTRACT

The present disclosure provides transaminase enzymes having improved properties and are capable of reducing (1S,5R)-6,8-dioxabicyclo[3.2.1]octan-4-one with high selectivity. Also provided are polynucleotides encoding the transaminase enzymes, host cells capable of expressing the transaminase enzymes, and methods of using the transaminase enzymes to synthesize (1S,4R,5R)-6,8-dioxabicyclo[3.2.1]octan-4-amine.

### SEQUENCE LISTING

The instant application contains a Sequence Listing which has been submitted electronically in ASCII format and is hereby incorporated by reference in its entirety. Said ASCII copy, created on May 25, 2022, is named 25246-WO-PCT_SL.txt and is 35,879 bytes in size.

### FIELD OF THE INVENTION

The present invention relates to transaminase enzymes, useful in biocatalytic and synthetic processes involving the reduction of ketones to chiral amines. Such enzymes may be particularly useful in synthetic processes that may be used to prepare (1S,4R,5R)-6,8-dioxabicyclo[3.2.1]octan-4-amine or similar compounds formed during preparation of such compounds.

### BACKGROUND OF THE INVENTION

Enzymes are protein molecules produced by organisms that catalyze biochemical transformations. Without enzymes, most biochemical reactions would be too slow to even carry out critical life processes. Enzymes in nature display great specificity for their cognate substrates and are not permanently modified by their participation in reactions. Because they are not changed during the reactions, enzymes can be cost effectively used as catalysts for a desired chemical transformation.

Transaminases are enzymes capable of catalyzing the reduction of ketones to amines in a selective manner. This biocatalytic transformation requires an amine donor, a carbonyl group that acts as the amine acceptor, and is mediated by pyridoxal-5′-phosphate (PLP). Transaminases are stereoselective enzymes, and their selectivity is modulated by the amino acids found within the active site. Transaminase enzymes are well known in nature, and numerous genes that encode transaminase enzymes and transaminase enzyme sequences have been reported. See, Kelly, Appl. Microbiol. Biotechnol. (2020) 104:4781-4794 and Guo, Green Chem., 2017, 19, 333 for comprehensive reviews.

Use of transaminase enzymes for synthetic commercial applications has attracted much attention over the past two decades due to the high utility of the transformations they carry out and the value of the chiral amine products. However, given the highly specific nature of enzyme catalysis, existing enzymes are typically not highly active or selective toward novel substrates. Wild-type or existing transaminases also typically display poor tolerance to heat and organic solvent, features that are often required for their use as industrial catalysts. The present invention is directed to transaminase variants having improved enzyme stability, selectivity, and activity for the formation of (1S,4R,5R)-6,8-dioxabicyclo[3.2.1]octan-4-amine from (1S,5R)-6,8-dioxabicyclo[3.2.1]octan-4-one, also known as Cyrene.

### SUMMARY OF THE INVENTION

The present disclosure relates to transaminase enzymes capable of converting ketones to amines in the presence of an isopropylamine donor and a cofactor. In embodiments, the subject transaminase enzymes described herein are capable of converting (1S,5R)-6,8-dioxabicyclo[3.2.1]octan-4-one to the corresponding amine, (1S,4R,5R)-6,8-dioxabicyclo[3.2.1]octan-4-amine. Furthermore, the subject transaminase enzymes described herein may be useful in the preparation of (3R,6S)-6-(hydroxymethyl)oxan-3-amine. Such compounds may be useful as intermediates for the synthesis of more complex biologically active compounds.

Additional embodiments describe processes for preparing the subject transaminase enzymes and processes for using the subject transaminase enzymes.

Other embodiments, aspects, and features of the present invention are either further described in or will be apparent from the ensuing description, examples and appended claims.

### DETAILED DESCRIPTION OF THE INVENTION

### Transaminases

The present invention relates to transaminase enzymes capable of catalyzing the reduction of ketones to amines in the synthesis of (1S,4R,5R)-6,8-dioxabicyclo[3.2.1]octan-4-amine. In the embodiments, the transaminase enzymes are capable of the following conversion:

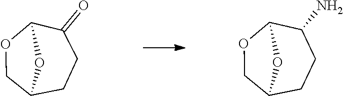 [SMILES: O=C1CC[C@H]2CO[C@@H]1O2 ; N[C@@H]1CC[C@H]2CO[C@@H]1O2]

In the embodiments, the transaminase enzymes described herein have an amino acid sequence that has one or more amino acid differences, as compared to a reference transaminase amino acid sequence, that result in an improved property of the enzyme with respect to carrying out the transamination of Cyrene. The transaminase enzymes described herein are the product of directed evolution from a commercially available transaminase, Enzyme 1, which is described in Yasuda, N. et al. Org. Process Res. Dev. 2017, 21, 1851-1858, WO201099501, WO2013036861 and U.S. Pat. No. 9,109,209 B2 which disclose the amino acid sequence as set forth below for SEQ ID NO: 1

(SEQ ID NO: 1)
MAFSADTPEIVYTHDTGLDYITYSDYELDPANPLAGGAAWIEGAFV
PPSEARISIFDQGFYTSDATYTVFHVWNGNAFRLGDHIERLFSNA
ESIRLIPPLTQDEVKEIALELVAKTELREAIVWVAITRGYSSTPL
ERDVTKHRPQVYMYAVPYQWIVPFDRIRDGVHLMVAQSVRRTPRS
SIDPQVKNFAAGDLIRAIQETHDRGFELPLLLDFDNLLAEGPGFN
VVVIKDGVVRSPGRAALPGITRKTVLEIAESLGHEAILADITPAE
LRDADEVLGCSTAGGVWPFVSVDGNSISDGVPGPVTQSIIRRYWE
LNVEPSCLLTPVQY

In specific occurrences, Enzyme 1 is encoded by the DNA sequence as set forth below in SEQ ID NO: 9

(SEQ ID NO: 9)
ATGGCGTTCTCAGCGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
CCGTCTGAAGCTCGTATCTCTATCTTCGACCAGGGTTTTTATACTTC
TGACGCTACCTACACCGTCTTCCACGTTTGGAACGGTAACGCTTTCC
GTCTGGGGGACCACATCGAACGTCTGTTCTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATTGTTTGGGTTG
CAATCACCCGTGGTTACTCTTCTACCCCATTGGAGCGTGACGTCACC
AAACATCGTCCGCAGGTTTACATGTATGCTGTTCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGGCAGGTGACCTGATCCGTGCAATTCAGGAAACCCACGA
CCGTGGTTTCGAGTTACCGCTGCTGCTGGACTTTGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTGAATCTCTGGGTCACGAAGCTATCCTGGCTGACA
TCACCCCGGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TACTAA

In embodiments, transaminase enzymes of the disclosure may demonstrate improvements relative the transaminase enzyme of SEQ ID NO: 1, such as increases in enzyme activity, diastereoselectivity, or thermostability.

In embodiments, the transaminase enzymes of the disclosure may demonstrate improvements in the rate of enzymatic activity, i.e., the rate of converting the substrate to the product. In some embodiments, the transaminase enzymes are capable of converting the substrate to the product at a rate that is at least 1.1 times the rate exhibited by the enzyme of SEQ ID NO: 1.

In some embodiments, such transaminase enzymes are also capable of converting the substrate to the product with a diastereometric ratio of at least 15:1. In some embodiments, such transaminase enzymes are also capable of converting the substrate to the product with a diastereometric ratio of at least 25:1. In some embodiments, such transaminase enzymes are also capable of converting the substrate to the product with a diastereometric ratio of at least 70:1.

In some embodiments, the transaminase enzyme is highly stereoselective, wherein the enzymes can reduce the substrate to the product in greater than about 50:1, 60:1 and 70:1 diastereometric ratio.

In some embodiments, the transaminase enzyme is highly thermostable, wherein the enzyme retains enzymatic activity at temperatures greater than 71° C. In some embodiments, the transaminase enzyme retains enzymatic activity at temperatures greater than 80° C.

In embodiments of the invention, the transaminase enzymes described herein include Enzyme 2 having the amino acid sequence as set forth below in SEQ ID NO: 2

(SEQ ID NO: 2)
MAFSLDTPEIVYTHDTGLDYITYSDYELDPANPLAGGAAWIEGAFVP
PSEARISIFDQGFYTSDATYTVFHVWNGNAFRLGDHIERLFSNAESI
RLIPPLTQDEVKEIALELVAKTELREAIVWVAITRGYSSTPLERDVT
KHRPQVYMYAVPYQWIVPFDRIRDGVHLMVAQSVRRTPRSSIDPQVK
NFAAGDLIRAIQETHDRGFELPLLLDFDNLLAEGPGFNVVVIKDGVV
RSPGRAALPGITRKTVLEIAESLGHEAILADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 2, having the DNA sequence as set forth below in SEQ ID NO: 10.

(SEQ ID NO: 10)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
CCGTCTGAAGCTCGTATCTCTATCTTCGACCAGGGTTTTTATACTTC
TGACGCTACCTACACCGTCTTCCACGTTTGGAACGGTAACGCTTTCC
GTCTGGGGGACCACATCGAACGTCTGTTCTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATTGTTTGGGTTG
CAATCACCCGTGGTTACTCTTCTACCCCATTGGAGCGTGACGTCACC
AAACATCGTCCGCAGGTTTACATGTATGCTGTTCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGGCAGGTGACCTGATCCGTGCAATTCAGGAAACCCACGA
CCGTGGTTTCGAGTTACCGCTGCTGCTGGACTTTGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTGAATCTCTGGGTCACGAAGCTATCCTGGCTGACA
TCACCCCGGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TACTAA

In additional embodiments, the transaminase enzymes described herein include transaminase Enzyme 3 having the amino acid sequence as set forth below in SEQ ID NO: 3.

(SEQ ID NO: 3)
MAFSLDTPEIVYTHDTGLDYITYSDYELDPANPLAGGAAWIEGAFVP
PSEARISVFDQGFYTSDATYTVFHVWNGNAFRLGDHIERLFSNAESI
RLIPPLTQDEVKEIALELVAKTELREAMVWVAITRGYSSTPLERDVT
KHRPQVYMYAVPYQWIVPFDRIRDGVHLMVAQSVRRTPRSSIDPQVK
NFAAGDLIRAIQETHDRGFELPLLLDHDNLLAEGPGFNVVVIKDGVV
RSPGRAALPGITRKTVLEIARSLGHEAILADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 3, having the DNA sequence as set forth below in SEQ ID NO: 11.

(SEQ ID NO: 11)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
CCGTCTGAAGCTCGTATCTCTGTTTTCGACCAGGGTTTTTATACTTC
TGACGCTACCTACACCGTCTTCCACGTTTGGAACGGTAACGCTTTCC
GTCTGGGGGACCACATCGAACGTCTGTTCTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATGGTTTGGGTTG
CAATCACCCGTGGTTACTCTTCTACCCCATTGGAGCGTGACGTCACC
AAACATCGTCCGCAGGTTTACATGTATGCTGTTCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGGCGGGCGACCTGATCCGTGCAATTCAGGAAACCCACGA
CCGTGGTTTCGAGTTACCGCTGCTGCTGGACCACGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTCGTTCTCTGGGTCACGAAGCTATTCTGGCTGACA
TCACCCCGGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TAC

In additional embodiments, the transaminase enzymes described herein include transaminase Enzyme 4 having the amino acid sequence as set forth below in SEQ ID NO: 4.

(SEQ ID NO: 4)
MAFSLDTPEIVYTHDTGLDYITYSDYELDPANPLAGGAAWIEGAFVP
PSEARISVFDQGFYTSDATYTVFHVWNGNAFRLGDHIERLFSNAESI
RLIPPLTQDEVKEIALELVAKTELREAMVWVAITRGYSSTPLERDVT
KHRPQVYMYAVPYQWIVPFDRIRDGVHLMVAQSVRRTPRSSIDPQVK
NFASIDLIRAIQETHDRGFELPLLLDHDNLLAEGPGFNVVVIKDGVV
RSPGRAALPGITRKTVLEIAESLGHEAMLADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 4, having the DNA sequence as set forth below in SEQ ID NO: 12.

(SEQ ID NO: 12)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
CCGTCTGAAGCTCGTATCTCTGTTTTCGACCAGGGTTTTTATACTTC
TGACGCTACCTACACCGTCTTCCACGTTTGGAACGGTAACGCTTTCC
GTCTGGGGGACCACATCGAACGTCTGTTCTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATGGTTTGGGTTG
CAATCACCCGTGGTTACTCTTCTACCCCATTGGAGCGTGACGTCACC
AAACATCGTCCGCAGGTTTACATGTATGCTGTGCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGTCGATTGACCTGATCCGTGCAATTCAGGAAACCCACGA
CCGTGGTTTCGAGTTACCGCTGCTGCTGGACCACGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTGAGTCTCTGGGTCACGAAGCTATGCTGGCTGACA
TCACCCCGGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TAC

In additional embodiments, the transaminase enzymes described herein include transaminase Enzyme 5 having the amino acid sequence as set forth below in SEQ ID NO: 5.

(SEQ ID NO: 5)
MAFSLDTPEIVYTHDTGLDYITYSDYELDPANPLAGGAAWIEGAFVP
PSEARISVFDQGFYTSDATYTAFHVWNGNAFRLGDHIERLFSNAESI
RLIPPLTQDEVKEIALELVAKTELREAMVWVAITRGYSSTPLERDVT
KHRPQVYMYAVPYQWIVPFDRIRDGVHLMVAQSVRRTPRSSIDPQVK
NFASIDLIRAIQETHDRGFELPLLLDHDNLLAEGPGFNVVVIKDGVV
RSPGRAALPGITRKTVLEIAESLGHEAMLADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 5, having the DNA sequence as set forth below in SEQ ID NO: 13.

(SEQ ID NO: 13)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
CCGTCTGAAGCTCGTATCTCTGTTTTCGACCAGGGTTTTTATACTTC
TGACGCTACCTACACCGCTTTCCACGTTTGGAACGGTAACGCTTTCC
GTCTGGGGGACCACATCGAACGTCTGTTCTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATGGTTTGGGTTG
CAATCACCCGTGGTTACTCTTCTACCCCATTGGAGCGTGACGTCACC
AAACATCGTCCGCAGGTTTACATGTATGCTGTGCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGTCGATTGACCTGATCCGTGCAATTCAGGAAACCCACGA
CCGTGGTTTCGAGTTACCGCTGCTGCTGGACCACGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTGAGTCTCTGGGTCACGAAGCTATGCTGGCTGACA
TCACCCCGGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TACTAA

In additional embodiments, the transaminase enzymes described herein include transaminase Enzyme 6 having the amino acid sequence as set forth below in SEQ ID NO: 6.

(SEQ ID NO: 6)
MAFSLDTPEIVYTHDTGLDYITYSDYELDPANPLAGGAAWIEGAFVP
VSEARISVFDQGFYASDATYTAFHVWNGNAFRLGDHIERLWSNAESI
RLIPPLTQDEVKEIALELVAKTELREAMVGVAITRGYSSTPLERDVT
KHRPQVYMYAVPYQWIVPFDRIRDGVHLMVAQSVRRTPRSSIDPQVK
NFASIDLIRAIQETHDRGFELPLLLDHDNLLAEGPGFNVVVIKDGVV
RSPGRAALPGITRKTVLEIARSLGHEAMLADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 6, having the DNA sequence as set forth below in SEQ ID NO: 14.

(SEQ ID NO: 14)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
GTCTCTGAAGCTCGTATCTCTGTTTTCGACCAGGGTTTTTATGCCTC
TGACGCTACCTACACCGCTTTCCACGTTTGGAACGGTAACGCTTTCC
GTCTGGGGGACCACATCGAACGTCTGTGGTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATGGTTGGGGTTG
CAATCACCCGTGGTTACTCTTCTACCCCATTGGAGCGTGACGTCACC
AAACATCGTCCGCAGGTTTACATGTATGCTGTCCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGTCGATTGACCTGATCCGTGCAATTCAGGAAACCCACGA
CCGTGGTTTCGAGTTACCGCTGCTGCTGGACCACGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTCGTTCTCTGGGTCATGAAGCTATGCTGGCTGACA
TCACCCCAGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TACTAA

In additional embodiments, the transaminase enzymes described herein include transaminase Enzyme 7 having the amino acid sequence as set forth below in SEQ ID NO: 7.

(SEQ ID NO: 7)
MAFSLDTPEIVYTHDTGLDYITYSDYELDPANPLAGGAAWIEGAFVP
VSEARISVFDQGFYASDATYTAFHVWNGNAFRLGDHIERLWSNAESI
RLIPPLTQDEVKEIALELVAKTELREAMVGVVITRGYSSTPLERDVT
KHRPQVYMYAIPYQWIVPFDRIRDGVHLMVAQSVRRTPRSSIDPQVK
NFASIDLIRAIQETHDRGFELPLLLDHDNLLAEGPGFNVVVIKDGVV
RSPGRAALPGITRKTVLEIARSLGHEAMLADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 7, having the DNA sequence as set forth below in SEQ ID NO: 15.

(SEQ ID NO: 15)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
GTCTCTGAAGCTCGTATCTCTGTTTTCGACCAGGGTTTTTATGCCTC
TGACGCTACCTACACCGCTTTCCACGTTTGGAACGGTAACGCTTTCC
GTCTGGGGGACCACATCGAACGTCTGTGGTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATGGTTGGGGTTG
TAATCACCCGTGGTTACTCTTCTACCCCATTGGAGCGTGACGTCACC
AAACATCGTCCGCAGGTTTACATGTATGCTATTCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGTCGATTGACCTGATCCGTGCAATTCAGGAAACCCACGA
CCGTGGTTTCGAGTTACCGCTGCTGCTGGACCACGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTCGTTCTCTGGGTCATGAAGCTATGCTGGCTGACA
TCACCCCAGCTGAACTGCGTGATGCCGACGAAGTTCTGGGTTGCTCA
ACCGCGGGTGGTGTTTGGCCGTTCGTTTCTGTTGACGGTAACTCGAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TACTAA

In additional embodiments, the transaminase enzymes described herein include transaminase Enzyme 8 having the amino acid sequence as set forth below in SEQ ID NO: 8.

(SEQ ID NO: 8)
MAFSLDTPEIVYTHDTGLDYITYSDYELDPANPLAGGAAWIEGAFVP
PSEARISVFDQGFYTSDATYTAFHVWNGNAFRLGDHIERLFSNAESI
RLIPPLTQDEVKEIALELVAKTELREAMVWVAITRGYSSTPLERDVT
KHRPQVYMYAVPYQWIVPFDRIRDGVHLMVAQSVRRTPRSSIDPQVK
NFAAGDLIRAIQETHDRGFELPLLLDHDNLLAEGPGFNVVVIKDGVV
RSPGRAALPGITRKTVLEIARSLGHEAILADITPAELRDADEVLGCS
TAGGVWPFVSVDGNSISDGVPGPVTQSIIRRYWELNVEPSCLLTPVQ
Y

The instant invention is also directed to a polynucleotide encoding the transaminase Enzyme 8 having the DNA sequence as set forth below in SEQ ID NO: 16.

(SEQ ID NO: 16)
ATGGCGTTCTCACTGGACACCCCTGAAATCGTTTACACCCACGACAC
CGGTCTGGACTATATCACCTACTCTGACTACGAACTGGACCCGGCTA
ACCCGCTGGCTGGTGGTGCCGCTTGGATCGAAGGTGCTTTCGTTCCG
CCGTCTGAAGCTCGTATCTCTGTTTTCGACCAGGGTTTTTATACTTC
TGACGCTACCTACACCGCTTTCCACGTTTGGAACGGTAACGCTTTCC
GTCTGGGGGACCACATCGAACGTCTGTTCTCTAATGCGGAATCTATT
CGTTTGATCCCGCCGCTGACCCAGGACGAAGTTAAAGAGATCGCTCT
GGAACTGGTTGCTAAAACCGAATTGCGTGAAGCGATGGTTTGGGTTG
CAATCACCCGTGGTTACTCTTCTACCCCATTGGAGCGTGACGTCACC
AAACATCGTCCGCAGGTTTACATGTATGCTGTTCCGTACCAGTGGAT
CGTACCGTTTGACCGCATCCGTGACGGTGTTCACCTGATGGTTGCTC
AGTCAGTTCGTCGTACACCGCGTAGCTCTATCGACCCGCAGGTTAAA
AACTTCGCGGCGGGCGACCTGATCCGTGCAATTCAGGAAACCCACGA
CCGTGGTTTCGAGTTACCGCTGCTGCTGGACCACGACAACCTGCTGG
CTGAAGGTCCGGGTTTCAACGTTGTTGTTATCAAAGACGGTGTTGTT
CGTTCTCCGGGTCGTGCTGCTCTGCCGGGTATCACCCGTAAAACCGT
TCTGGAAATCGCTCGTTCTCTGGGTCACGAAGCTATTCTGGCTGACA
TCACCCCGGCTGAACTGCGTGATGCCGACGAAGTTCTGGGTTGCTCA
ACCGCGGGTGGTGTTTGGCCGTTCGTTTCTGTTGACGGTAACTCTAT
CTCTGACGGTGTTCCGGGTCCGGTTACCCAGTCTATCATCCGTCGTT
ACTGGGAACTGAACGTTGAACCTTCTTGCCTGCTGACCCCGGTACAG
TACTAA

The transaminase enzymes of the disclosure are based on the amino acid sequences of SEQ ID NO: 2, 3, 4, 5, 6, 7 or 8 and can comprise an amino acid sequence that has substantial identity to the reference sequence of SEQ ID NO: 2, 3, 4, 5, 6, 7 or 8. In other embodiments, the transaminase enzyme can comprise an amino acid sequence that is at least about 90%, 91%, 92%, 93%, 94%, 95%, 96%, 97%, 98% or 99% identical to the reference sequence of SEQ ID NO: 2, 3, 4, 5, 6, 7 or 8. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

Embodiments of the invention are directed to transaminase enzymes comprising an amino acid sequence having at least a 90% sequence identity to SEQ ID NO: 2, 3, 4, 5, 6, 7 or 8.

The instant invention is also directed to polynucleotides encoding the transaminase enzymes of SEQ ID NO: 2, 3, 4, 5, 6, 7 or 8. In an embodiment of the invention, the polynucleotides can comprise DNA sequences that are at least about 90%, 91%, 92%, 93%, 94%, 95%, 96%, 97%, 98% or 99% identical to the reference sequence of SEQ ID NO: 10, 11, 12, 13, 14, 15, or 16.

### Definitions

Terms used herein have their ordinary meaning, which is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates.

As used herein, and throughout this disclosure, the following terms, unless otherwise indicated, shall be understood to have the following meanings:

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

As used herein, the singular forms “a”, “an” and “the” include plural referents unless the context clearly indicates otherwise. Thus, for example, reference to “a polypeptide” includes more than one polypeptide.

Similarly, “comprise,” “comprises,” “comprising” “include,” “includes,” and “including” are interchangeable and not intended to be limiting. Thus, as used herein, the term “comprising” and its cognates are used in their inclusive sense (i.e., equivalent to the term “including” and its corresponding cognates).

It is to be further understood that where descriptions of various embodiments use the term “comprising,” those skilled in the art would understand that in some specific instances, an embodiment can be alternatively described using language “consisting essentially of” or “consisting of.”

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

As used herein, “ATCC” refers to the American Type Culture Collection whose biorepository collection includes genes and strains.

“Protein,” “polypeptide,” and “peptide” are used interchangeably herein to denote a polymer of at least two amino acids covalently linked by an amide bond, regardless of length or post-translational modification (e.g., glycosylation or phosphorylation). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of a transaminase polypeptide can be expressed as “percent conversion” of the substrate to the product.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. MOL. BIOL. 215: 403-410; and Altschul et al., 1977, NUCLEIC ACIDS RES. 3389-3402). Software for performing BLAST analyses is publicly available through the National Center for Biotechnology Information website.

Numerous other algorithms are available that function similarly to BLAST in providing percent identity for two sequences. Optimal alignment of sequences for comparison can be conducted, e.g., by the local homology algorithm of Smith and Waterman, 1981, ADV. APPL. MATH. 2:482, by the homology alignment algorithm of Needleman and Wunsch, 1970, J. MOL. BIOL. 48:443, by the search for similarity method of Pearson and Lipman, 1988, N USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

As used herein, “polynucleotide” and “nucleic acid’ are used interchangeably and refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides, and may include a DNA or RNA of genomic, mRNA, cDNA, or synthetic origin, or some combination thereof.

While the nucleosides will typically be linked together via standard phosphodiester linkages, the polynucleotides may include one or more non-standard linkages. The polynucleotide may be single-stranded or double-stranded, or the polynucleotide may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

As used herein, the terms “biocatalysis,” “biocatalytic,” “biotransformation,” and “biosynthesis” refer to the use of enzymes to perform chemical reactions on organic compounds.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acid having an aromatic side chain is substituted with another amino acid having an aromatic side chain (e.g., phenylalanine, tyrosine, tryptophan, and histidine); an amino acid with a basic side chain is substituted with another amino acid with a basic side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide that is substantially separated from other contaminants that naturally accompany it (e.g., protein, lipids, and polynucleotides). The term embraces polypeptides that have been removed or purified from their naturally occurring environment or expression system (e.g., within a host cell or via in vitro synthesis). The recombinant polypeptides may be present within a cell, present in the cellular medium, or prepared in various forms, such as lysates or isolated preparations. As such, in some embodiments, the recombinant polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, an enzyme comprising composition comprises enzymes that are less than 50% pure (e.g., about 10%, about 20%, about 30%, about 40%, or about 50%). Generally, a substantially pure enzyme or polypeptide composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500) Daltons), and elemental ion species are not considered macromolecular species. In some embodiments, the isolated recombinant polypeptides are substantially pure polypeptide compositions.

“Improved enzyme property” refers to an enzyme that exhibits an improvement in any enzyme property as compared to a reference enzyme. For the enzymes described herein, the comparison is generally made to the transaminase Enzyme 1, although in some embodiments, the reference enzyme can be another improved enzyme. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity and diastereoselectivity).

“Increased enzymatic activity” refers to an improved property of the enzymes, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. Exemplary methods to determine enzyme activity are provided in the Examples. Any property relating to enzyme activity may be affected, including the classical enzyme properties of Kₘ, Vₘₐₓ, or k_{cat}, changes of which can lead to increased enzymatic activity. Improvements in enzyme activity can be from about 1.1 times, about 1.5 times, or about 2 times the enzymatic activity of the corresponding parent enzyme from which the polypeptide is derived.

It is understood by the skilled artisan that the activity of any enzyme is diffusion limited such that the catalytic turnover rate cannot exceed the diffusion rate of the substrate, including any required cofactors. The theoretical maximum of the diffusion limit, or k_{cat}/Kₘ, is generally about 108 to 109 (M⁻¹s⁻¹). Hence, any improvements in the enzyme activity will have an upper limit related to the diffusion rate of the substrates acted on by the enzyme. Enzyme activity can be measured by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

Additional embodiments provide host cells containing the polynucleotides and/or expression vectors described herein. The host cells may be E. coli, or they may be a different organism. The host cells can be used for the expression and isolation of the transaminase enzymes described herein, or, alternatively, they can be used directly for the conversion of the substrate to the stereoisomeric product.

Whether carrying out the method with whole cells, cell extracts or purified transaminase enzymes, a single transaminase enzyme may be used or, alternatively, mixtures of two or more transaminases enzymes may be used.

Embodiments relate to transaminase enzymes capable of selectively preparing (1S,4R,5R)-6,8-dioxabicyclo[3.2.1]octan-4-amine in the synthesis of (3R,6S)-6-(hydroxymethyl)oxan-3-amine. In embodiments, the transaminase enzymes are capable of the following conversion:

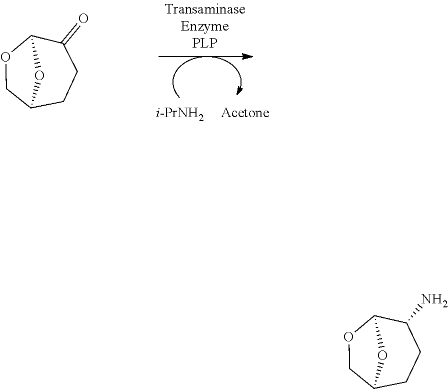 [SMILES: "i-PrNH2" ; O=C1CC[C@H]2CO[C@@H]1O2 ; N[C@@H]1CC[C@H]2CO[C@@H]1O2]

The process of using the transaminase enzymes of the instant invention is described in a separate, commonly owned, co-filed patent application, incorporated by reference in its entirety.

### Polynucleotides Encoding Transaminases

In another aspect, the present disclosure provides polynucleotides encoding the transaminase enzymes disclosed herein. The polynucleotides may be operatively linked to one or more heterologous regulatory sequences that control gene expression to create a recombinant polynucleotide capable of expressing the polypeptide. Expression constructs containing a heterologous polynucleotide encoding the transaminase can be introduced into appropriate host cells to express the corresponding transaminase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved transaminase enzymes disclosed herein. Thus, having identified a particular amino acid sequence, those skilled in the art could make any number of different nucleic acids by simply modifying the sequence of one or more codons in a way that does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. By way of example, the polynucleotides of SEQ ID NO: 1 have been codon optimized for expression in Escherichia coli.

In certain embodiments, all codons need not be replaced to optimize the codon usage of the transaminase enzyme since the natural sequence will comprise preferred codons and because use of preferred codons may not be required for all amino acid residues. Consequently, codon optimized polynucleotides encoding the transaminase enzymes may contain preferred codons at about 40%, 50%, 60%, 70%, 80%, or greater than 90% of codon positions of the full-length coding region.

In various embodiments, an isolated polynucleotide encoding an improved transaminase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, MOLECULAR CLONING: A LABORATORY MANUAL, 3rd Ed., Cold Spring Harbor Laboratory Press; and CURRENT PROTOCOLS IN MOLECULAR BIOLOGY, Ausubel. F. ed., Greene Pub. Associates, 1998, updates to 2006.

In some embodiments, an isolated polynucleotide encoding any of the transaminase polypeptides herein is manipulated in a variety of ways to facilitate expression of the transaminase polypeptide. In some embodiments, the polynucleotides encoding the transaminase polypeptides comprise expression vectors where one or more control sequences is present to regulate the expression of the transaminase polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators. In some embodiments, suitable promoters are selected based on the host cell selection. For bacterial host cells, suitable promoters for directing transcription of the nucleic acid constructs of the present disclosure, include, but are not limited to, promoters obtained from the E. coli lac operon. In addition, suitable promotors may include Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., PROC. NATL ACAD. SCI. USA 75: 3727-3731 [1978]), as well as the tac promoter (See e.g., DeBoer et al., PROC. NATL ACAD. SCI. USA 80: 21-25 [1983]).

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the enzyme polypeptide. Any suitable terminator that is functional in the host cell of choice finds use in the present invention.

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the transaminase polypeptide. Any suitable leader sequence that is functional in the host cell of choice find use in the present invention. Exemplary leaders for E. coli will encode a ribosome binding site.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to, the lac, tac, and trp operator systems. In yeast host cells, suitable regulatory systems include, but are not limited to, the ADH2 system or GAL1 system. In filamentous fungi, suitable regulatory sequences include, but are not limited to, the TAKA alpha-amylase promoter, Aspergillus niger glucoamylase promoter, and Aspergillus oryzae glucoamylase promoter.

In another aspect, the present invention is directed to a recombinant expression vector comprising a polynucleotide encoding transaminase polypeptide, and one or more expression regulating regions such as a promoter and a terminator, a replication origin, etc., depending on the type of hosts into which they are to be introduced. In some embodiments, the various nucleic acid and control sequences described herein are joined together to produce recombinant expression vectors that include one or more convenient restriction sites to allow for insertion or substitution of the nucleic acid sequence encoding the enzyme polypeptide at such sites. Alternatively, in some embodiments, the nucleic acid sequence of the present invention is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the enzyme polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to, the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to, ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present invention provides a host cell comprising at least one polynucleotide encoding at least one transaminase of the present invention, the polynucleotide(s) being operatively linked to one or more control sequences for expression of the at least one transaminase in the host cell. Host cells suitable for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)). Exemplary host cells also include various Escherichia coli strains (e.g., W3110 (AfhuA) and BL21). Examples of bacterial selectable markers include, but are not limited to, the dal genes from Bacillus subtilis or Bacillus licheniformis, or markers, which confer antibiotic resistance such as ampicillin, kanamycin, chloramphenicol, and or tetracycline resistance.

In some alternative embodiments, the expression vectors contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements preferably contain a sufficient number of nucleotides, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori or the origins of replication of plasmids pBR322, pUC19, pACYCI77 (which contains the P15A ori), or pACYC184 (which contains the P15A ori) permitting replication in E. coli, and pUB110, pE194, or pTA1060 permitting replication in Bacillus. Examples of origins of replication for use in a yeast host cell are the 2 micron origin of replication, ARSI, ARS4, the combination of ARSI and CEN3, and the combination of ARS4 and CEN6. The origin of replication may be one having a mutation which makes its functioning temperature-sensitive in the host cell (See e.g., Ehrlich, Proc. Natl. Acad. Sci. USA 75:1433) [1978].

In some embodiments, more than one copy of a nucleic acid sequence of the present invention is inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present invention are commercially available. Suitable commercial expression vectors include, but are not limited to, Novagen's R pET E. coli T7 expression vectors (Millipore Sigma) and the p3×FLAG™™ expression vectors (Sigma-Aldrich Chemicals). Other suitable expression vectors include, but are not limited to, pBluescriptII SK(−) and pBK-CMV (Stratagene), and plasmids derived from pBR322 (Gibco BRL), pUC (Gibco BRL), pREP4, pCEP4 (Invitrogen) or pPoly (See e.g., Lathe et al., Gene 57:193-201 [1987]).

Thus, in some embodiments, a vector comprising a sequence encoding at least one variant transaminase is transformed into a host cell in order to allow propagation of the vector and expression of the variant transaminase(s). In some embodiments, the transformed host cell described above is cultured in a suitable nutrient medium under conditions permitting the expression of the variant transaminase(s). Any suitable medium useful for culturing the host cells finds use in the present invention, including, but not limited to minimal or complex media containing appropriate supplements. In some embodiments, host cells are grown in HTP media. Suitable media are available from various commercial suppliers or may be prepared according to published recipes (e.g., in catalogues of the American Type Culture Collection).

### Host Cells for Expression of Transaminases

In another aspect, the present disclosure provides a host cell comprising a polynucleotide encoding an improved transaminase polypeptide of the present disclosure, the polynucleotide being operatively linked to one or more control sequences for expression of the transaminase enzyme in the host cell. Host cells for use in expressing the transaminase polypeptides encoded by the expression vectors of the present invention are well known in the art and include but are not limited to, bacterial cells, such as E. coli, B. subtilis, B. licheniformis, B. megaterium, B. stearothermophilus, B. amyloliquefaciens, Klebsiella aerogenes, Lactobacillus kejir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)). Appropriate culture mediums and growth conditions for the above-described host cells are well known in the art.

Polynucleotides for expression of the transaminase may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

In some embodiments of the present invention, the filamentous fungal host cells are of any suitable genus and species, including, but not limited to Achlya, Acremonium, Aspergillus, Aureobasidium, Bjerkandera, Ceriporiopsis. Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum. Talaromyces, Thermoascus, Thielavia, Trametes, Tolypocladium, Trichoderma, Verticillium, and/or Volvariella, and/or teleomorphs, or anamorphs, and synonyms, basionyms, or taxonomic equivalents thereof.

In some embodiments of the present invention, the host cell is a yeast cell, including but not limited to cells of Candida, Hansenula, Saccharomyces, Schizosaccharomyces, Pichia, Kluyveromyces, or Yarrowia species. In some embodiments of the present invention, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria. Pichia quercuum. Pichia pijperi, Pichia stipitis, Pichia methanolica, Pichia angusta, Kluyveromyces lactis, Candida albicans, or Yarrowia lipolytica.

In some other embodiments, the host cell is a prokaryotic cell. Suitable prokaryotic cells include, but are not limited to, Gram-positive, Gram-negative and Gram-variable bacterial cells. Any suitable bacterial organism finds use in the present invention, including but not limited to Agrobacterium, Alicyclobacillus, Anabaena, Anacystis. Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas. In some embodiments, the bacterial host strain is non-pathogenic to humans. In some embodiments the bacterial host strain is an industrial strain. Numerous bacterial industrial strains are known and suitable in the present invention. In some embodiments of the present invention, the bacterial host cell is an Agrobacterium species (e.g., A. radiobacter, A. rhizogenes, and A. rubi). In some embodiments of the present invention, the bacterial host cell is an Arthrobacter species (e.g., A. aurescens, A. citreus, A. globiformis, A. hydrocarboglutamicus, A. mysorens, A. nicotianae, A. paraffineus, A. protophonniae, A. roseoparaffinus, A. sulfureus, and A. ureafaciens). In some embodiments of the present invention, the bacterial host cell is a Bacillus species (e.g., B. thuringensis, B. anthracis, B. megaterium, B. subtilis, B. lentus, B. circulans, B. pumilus, B. lautus, B. coagulans. B. brevis. B. firmus. B. alkaophius, B. licheniformis, B. clausii, B. stearothermophilus, B. halodurans, and B. amyloliquefaciens). In some embodiments, the host cell is an industrial Bacillus strain including but not limited to B. subtilis, B. pumilus, B. licheniformis, B. megaterium. B. clausii, B. stearothermophilus, or B. amyloliquefaciens. In some embodiments, the Bacillus host cells are B. subtilis. B. licheniformis, B. megaterium. B. stearothermophilus. and/or B. amyloliquefaciens. In some embodiments, the bacterial host cell is a Clostridium species (e.g., C. acetobutylicum, C. tetani E88, C. lituseburense, C. saccharobutylicum, C. perfringens, and C. beijerinckii). In some embodiments, the bacterial host cell is a Corynebacterium species (e.g., C. glutamicum and C. acetoacidophilum). In some embodiments the bacterial host cell is an Escherichia species (e.g., E. coli). In some embodiments, the host cell is Escherichia coli W3110. In some embodiments the host is Escherichia coli BL21 or BL21(DE3). In some embodiments, the bacterial host cell is an Erwinia species (e.g., E. uredovora, E. carotovora, E. ananas, E. herbicola, E. punctata, and E. terreus). In some embodiments, the bacterial host cell is a Pantoea species (e.g., P. citrea, and P. agglomerans). In some embodiments the bacterial host cell is a Pseudomonas species (e.g., P. putida, P. aeruginosa, P. mevalonii, and P. sp. D-01 10). In some embodiments, the bacterial host cell is a Streptococcus species (e.g., S. equisimiles, S. pyogenes, and S. uberis). In some embodiments, the bacterial host cell is a Streptomyces species (e.g., S. ambofaciens, S. achromogenes, S. avermitilis, S. coelicolor, S. aureofaciens. S. aureus, S. fungicidicus. S. griseus, and S. lividans). In some embodiments, the bacterial host cell is a Zymomonas species (e.g., Z. mobilis, and Z. lipolytica).

Many prokaryotic and eukaryotic strains that find use in the present invention are readily available to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of transaminase variant(s) within the host cell and/or in the culture medium. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression. A variety of methods are known in the art for reducing expression of protein in cells, including, but not limited to deletion of all or part of the gene encoding the protein and site-specific mutagenesis to disrupt expression or activity of the gene product. (See e.g., Chaveroche et al., NUCL. ACIDS RES., 28:22 e97 [2000]; Cho et al., MOLEC. PLANT MICROBE INTERACT., 19:7-15 [2006]; Maruyama and Kitamoto, BIOTECHNOL LETT., 30:1811-1817 [2008]; Takahashi et al., MOL. GEN. GENOM., 272: 344-352 [2004]; and You et al., Arch. Microbiol., 191:615-622 [2009], all of which are incorporated by reference herein). Random mutagenesis, followed by screening for desired mutations also finds use (See e.g., Combier et al., FEMS MICROBIOL. LETT., 220:141-8 [2003]; and Firon et al., EUKARY. CELL 2:247-55 [2003], both of which are incorporated by reference).

Introduction of a vector or DNA construct into a host cell can be accomplished using any suitable method known in the art, including but not limited to calcium phosphate transfection, DEAE-dextran mediated transfection, PEG-mediated transformation, electroporation, or other common techniques known in the art.

In some embodiments, the engineered host cells (i.e., “recombinant host cells”) of the present invention are cultured in conventional nutrient media modified as appropriate for activating promoters, selecting transformants, or amplifying the transaminase polynucleotide. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archebacterial origin.

In some embodiments, cells expressing the transaminase of the invention are grown under batch or continuous fermentation conditions. Classical “batch fermentation” is a closed system, wherein the compositions of the medium are set at the beginning of the fermentation and is not subject to artificial alternations during the fermentation. A variation of the batch system is a “fed-batch fermentation” that also finds use in the present invention. In this variation, nutrients, growth factors, or other supplements are added in increments as the fermentation progresses. Fed-batch systems are useful when catabolite repression is likely to inhibit the metabolism of the cells. Batch and fed-batch fermentations are common and well known in the art. “Continuous fermentation” is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

In some embodiments of the present invention, cell-free transcription and translation systems find use in producing the transaminases(s). Several systems are commercially available, and the methods are well-known to those skilled in the art.

### Methods of Evolving Transaminases

In some embodiments, to make the transaminase of the present disclosure, the transaminase enzyme that catalyzes the reduction reaction is obtained (or derived) from E. coli. In some embodiments, the parent polynucleotide sequence is codon-optimized to enhance expression of the transaminase in a specified host cell. The parental polynucleotide sequence, designated as SEQ ID NO: 1, was cloned into an expression vector, placing the expression of the transaminase gene under the control of the lac promoter under control of the lac repressor. Clones expressing the active transaminase in Escherichia coli were identified, and the genes sequenced to confirm their identity.

The transaminase of the disclosure may be obtained by subjecting the polynucleotide encoding the parent sequence to mutagenesis and/or directed evolution methods. An exemplary directed evolution technique is mutagenesis and/or DNA shuffling as described in Stemmer, 1994, PROC. NATL. ACAD. SCI. USA 91:10747-10751: WO 95/22625: WO 97/20078; WO 97/35966: WO 98/27230; WO 00/42651: WO 01/75767 and U.S. Pat. No. 6,537,746. Other directed evolution procedures that can be used include, among others, staggered extension process (StEP), in vitro recombination (Zhao et al., 1998, NAT. BIOTECHNOL. 16:258-261), mutagenic PCR (Caldwell et al., 1994, PCR METHODS APPL. 3:S136-S140), and cassette mutagenesis (Black et al., 1996, PROC. NATL. ACAD. SCI. USA 93:3525-3529).

The clones obtained following mutagenesis are screened for desired improvements in enzyme properties. Measuring enzyme activity and selectivity from the variant libraries can be performed using standard chemistry analytical techniques for measuring substrates and products such as UPLC, UPLC-MS, GC, or SFC. In this reaction, the amine of the isopropylamine donor is transferred to the (1S,5R)-6,8-dioxabicyclo[3.2.1]octan-4-one by the transaminase in the presence of a pyridoxal 5′-phosphate cofactor to yield acetone and (1S,4R,5R)-6,8-dioxabicyclo[3.2.1]octan-4-amine. The reaction may also be run under conditions where the transaminase is the yield-limiting catalyst, such that a doubling or halving in concentration of the transaminase will affect a doubling or halving of the yield of product observed at a given timepoint. Where the improved enzyme property desired is thermal stability, enzyme activity may be measured after subjecting the enzyme preparations to a defined temperature and measuring the amount of enzyme activity remaining after heat treatments. Clones containing a polynucleotide encoding improved transaminase variants are then isolated, sequenced to identify the nucleotide sequence changes, and used to express the enzyme in a host cell.

Where the sequence of the polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the invention can be prepared by chemical synthesis using, e.g., the classical phosphoramidite method described by Beaucage et al., 1981, TET. LETT. 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif., ExpressGen Inc. Chicago, Ill., Operon Technologies Inc., Alameda, Calif., and many others.

Transaminase enzymes expressed in a host cell can be recovered from the cells and/or the culture medium using any one or more of the well-known techniques for protein purification, including, among others, lysozyme treatment, sonication, filtration, salting-out, ultra-centrifugation, and chromatography.

Chromatographic techniques for isolation of the transaminase polypeptide include, among others, reverse phase chromatography, high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved transaminase enzymes. For affinity chromatography purification, the protein sequence can be tagged with a recognition sequence to enable purification. Common tags include cellulose-binding domains, poly His-tags, di-His chelates, FLAG-tags and many others that will be apparent to those having skill in the art. Antibodies can also be used as affinity purification reagents. Any antibody that specifically binds the transaminase polypeptide may be used.

### Methods of Using the Transaminases

The transaminase enzymes described herein can catalyze the reduction of substrate compounds, such as Compound 2 below

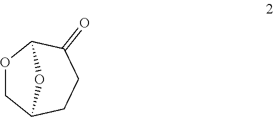

to the corresponding isomeric product, such as Compound 3 below:

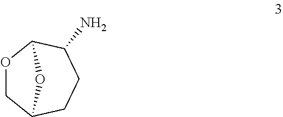

or a salt thereof.

As used herein, Compound 2 may also be referred to herein as Cyrene or (1S,5R)-6,8-Dioxabicyclo[3.2.1]octan-4-one. Compound 3 may also be referred to herein as (1S,4R,5R)-6,8-Dioxabicyclo[3.2.1]octan-4-amine.

In some embodiments, the method for reducing the substrate comprises contacting or incubating the substrate with a transaminase disclosed herein under conditions suitable for reaction. In some embodiments, the product is produced in 16:1, 17:1, 20:1, 25:1, 36:1, 39:1 44:1, 50:1, 60:1 or 73:1 diastereometric ratio over the corresponding minor product. In some embodiments, the product is produced in 17:1, 20:1, 25:1, 36:1, 44:1, 50:1, or 73:1 diastereometric ratio over the corresponding minor product.

In some embodiments of the method for reducing the substrate to the product, the substrate is reduced to give the product in greater than 16:1 diastereometric ratio, wherein the transaminase polypeptide comprises a sequence that corresponds to SEQ ID NO: 2, 3, 4, 5, 6, 7 or 8.

In another embodiment of this method for reducing the substrate to the product, at least about 40% of the substrate is converted to the product in less than about 24 hours when carried out with greater than about 75 g/L of substrate and less than about 15 g/L of the polypeptide, wherein the polypeptide comprises an amino acid sequence corresponding to SEQ ID NO: 2, 3, 4, 5, 6, 7 or 8. In another embodiment of this method for reducing the substrate to the product, at least about 50% of the substrate is converted to the product in less than about 24 hours when carried out with greater than about 75 g/L of substrate and less than about 15 g/L of the polypeptide, wherein the polypeptide comprises an amino acid sequence corresponding to SEQ ID NO: 2, 3, 4, 5, 6, 7 or 8.

As is known by those of skill in the art, transaminase-catalyzed reactions require a cofactor. As used herein, the term “cofactor” refers to a non-protein compound that operates in combination with a transaminase enzyme. Cofactors suitable for use with the engineered transaminase enzymes described herein include compounds from the vitamin B6 family, including, but not limited to, pyridoxal 5′-phosphate (PLP) or pyridoxamine 5′-phosphate (PMP).

The transaminase-catalyzed reactions described herein are generally carried out in a solvent, which may include, but not be limited to, aqueous solvents, including water and aqueous co-solvent systems. Examples of solvents that may be used are include aqueous or organic solvents, including water, isopropyl acetate, methyl-tetrahydrofuran, methyl t-butyl ether (MTBE), toluene, and the like can be used. In some instances, mixtures of aqueous and organic solvents are used, including but not limited to water and one or more organic solvent.

Whole cells transformed with gene(s) encoding the transaminase enzyme, or cell extracts and/or lysates thereof, may be employed in a variety of different forms, including solid (e.g., lyophilized, spray-dried, and the like) or semisolid (e.g., a crude paste).

The cell extracts or cell lysates may be partially purified by precipitation (ammonium sulfate, polyethyleneimine, heat treatment or the like), followed by a desalting procedure prior to lyophilization (e.g., ultrafiltration, dialysis, and the like). Any of the cell preparations may be stabilized by crosslinking using known crosslinking agents.

Suitable conditions for carrying out the transaminase-catalyzed reactions described herein include a wide variety of conditions that can be readily optimized by routine experimentation that includes, but is not limited to, contacting the engineered transaminase enzyme and substrate at an experimental pH and temperature and detecting product, for example, using the methods described in the Examples provided herein.

The reaction is generally allowed to proceed until essentially complete, or near complete, reduction of substrate is obtained. Reduction of substrate to product can be monitored using known methods by detecting substrate and/or product. Suitable methods include HPLC-CAD (charged aerosol detection), SFC-MS, HPLC-MS, GC, and the like. Product may also be detected after derivatization using known reagents for derivatization of amines. Conversion yields of the reduction product generated in the reaction mixture are generally greater than about 40%, may also be greater than about 50%, may also be greater than about 60%, and are often greater than 70%.

### Abbreviations

IPTG Isopropyl ß-D-1-thiogalactopyranoside
LB Luria-Bertani broth, commercially available, nutritionally rich medium, for culture and growth of bacteria
TB Terrific broth, commercially available, nutrionally rich medium for culture and growth of bacteria
pCK110900 Expression system of recombinant proteins in E. coli
PLP pyridoxal 5′-phosphate
OD600 Optical density at 600 nm
w/v Weight per volume

### EXAMPLES

### Example 1: Enzyme Preparation for Well Plate Reactions

LB-agar supplemented with 34 micrograms per mL chloroamphenicol and 1% (w/v) glucose in Q-tray plates was inoculated with a glycerol stock of E. coli W3110 strain cells harboring plasmid encoding for Transaminase Enzyme 1 in the pCK110900 vector (Enzyme 1 is commercially available from Codexis, Inc.). The plate was incubated at 37° C. overnight. The following day, a 96-well plate containing 0.2 mL per well of Luria-Bertani Broth (culture media for cells) supplemented with 34 micrograms per mL of chloroamphenicol and 1% (w/v) glucose was inoculated with individual colonies. The 96-well plate was shaken at 250 RPM/30° C. overnight. The following day, the cells' optical density at 600 nm (OD600) was measured, and an aliquot of cells was diluted to an OD600 of 0.05 in ˜390 uL of Terrific Broth media supplemented with 34 micrograms per mL of chloroamphenicol and 0.1 mM pyridoxine in a new 96-deep well plate. This culture was grown at 30° C./250 RPM for ˜2.5 hours until OD600 reached 0.6-0.8. Protein production was induced with 1 mM IPTG for 20 hours at 30° C./250 RPM. Cells were pelleted by centrifugation and the supernatants were discarded. Cell pellets were frozen, thawed, and then resuspended in lysis buffer (0.4 mL per well of 100 mM triethanolamine-HCl pH 7.5, 0.25 mg/mL lysozyme, 0.2 mg/mL polymyxin B sulfate, 1.6 U/mL DNaseI, and 0.1 mM PLP). The plate was then shaken for 2 h at room temperature at 1000 RPM. The lysate was then clarified by centrifugation (4000×g, 15 minutes). The supernatant following this step was then used in subsequent well-plate reactions (Example 3). This protocol was followed to prepare any transaminase enzyme of SEQ ID NO: 1 through SEQ ID NO: 8.

### Example 2: Enzyme Preparation as Lyophilized Cell-Free Lysate Powder

25 mL of LB broth supplemented with 34 micrograms per mL chloroamphenicol and 1% (w/v) glucose was inoculated with 20 microliters of a glycerol stock of E. coli W3110 strain cells harboring plasmid encoding for transaminase in the pCK110900 vector. Cells were grown until saturation for 18 hours at 30° C./250 RPM. The following day, a 2.8 L flask containing 1 L of TB supplemented with 34 micrograms per mL of chloroamphenicol and 0.1 mM pyridoxine was subcultured with the overnight saturated culture to an initial OD600 of 0.05. This culture was grown at 30° C./250 RPM for ˜2.5 hours until the OD600 reached 0.6-0.8. Protein production was induced with 1 mM IPTG for 20 hours at 30° C./250 RPM. Cells were pelleted by centrifugation and the supernatants were discarded. Cell pellets were flash frozen in liquid nitrogen, thawed and resuspended with 5 mL (per gram of pellet) of ice-cold 50 mM triethanolamine-HCl buffer pH 7.5 supplemented with 0.1 mM PLP. This suspension was shaken at 20° C. for 30 minutes, after which time the cells were placed on ice to chill, and then disrupted by high-pressure homogenization (16,000 PSI). The resulting lysate was then clarified by centrifuging at 10,000×g for 45 minutes at 4° C. Following centrifugation, the supernatant was frozen and lyophilized. This protocol was followed to prepare any transaminase enzyme of SEQ ID NO: 1 through SEQ ID NO: 8.

### Example 3: Transaminase Reactions in Well Plates

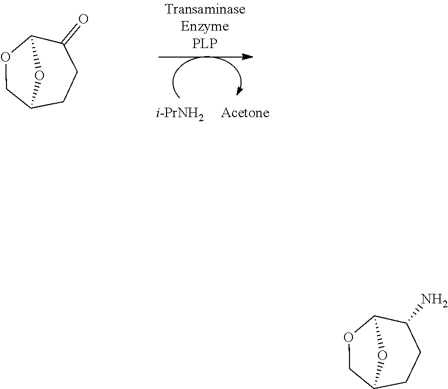 [SMILES: "i-PrNH2" ; O=C1CC[C@H]2CO[C@@H]1O2 ; N[C@@H]1CC[C@H]2CO[C@@H]1O2]

To each well of a 96-well plate were charged 1 g/L PLP, 1 M isopropylamine in 100 mM borate buffer pH 10.5, 50 g/L (1S,5R)-6,8-dioxabicyclo[3.2.1]octan-4-one, 0.5 g/L aniline, and 5% by volume lysate containing the transaminase enzyme with the amino acid sequence specified by SEQ ID NO: 6. The plate was sealed and shaken at 45° C., 1000 RPM overnight. 40 μL of the reaction mixture was then diluted with 160 μL 80/20 (v/v) acetonitrile:water containing 200 mM triethylamine and 27 mg/mL 1-fluoro-2,4-dinitrobenzene (DNFB) for derivatization. Derivatization reactions were aged for 1 h at room temperature at 600 RPM. The reactions were then quenched by diluting 5-fold into 80% acetonitrile, filtered, and analyzed by UPLC.

### Example 4: Transaminase Reactions Using Lyophilized Cell-Free Lysate

Cyrene (1.5 g, (1S,5R)-6,8-dioxabicyclo[3.2.1]octan-4-one) was dissolved in buffer (0.1 M borate, 1 M isopropylamine, pH 10.5, 10 mL) containing 1 mg/mL pyridoxal-5′-phosphate (PLP). Separately, transaminase enzyme (45 mg of lyophilized cell-free powder) was dissolved in buffer (2 mL, 0.1 M borate, 1 M isopropylamine, pH 10.5) containing 1 mg/ml PLP. In vials, 250 μL of the cyrene/PLP stock were combined with 250 μL of the transaminase enzyme/PLP stock. Reactions were heated to 45° C. with shaking. After 17 to 24 b, 100 μL of each reaction was diluted with 600 μL of 0.5% maleic acid in D20 for analysis by quantitative ¹H NMR. To determine selectivity, a stock of 1-fluoro-2,4-dinitrobenzene (DNFB, 150 mg) and triethylamine (200 μL) was prepared in 9.6 mL acetonitrile. In vials, 990 μL of this reagent mixture were combined with 20 μL of each reaction. After at least 4 h of incubation, the derivatization reactions were filtered and analyzed by UPLC. When the transaminase enzyme is enzyme 6, the reaction proceeded to 65% conversion after 18 hours with 73:1 dr, as seen in Table 1 below. The activity, selectivity, and thermostability of transaminase enzymes 1, 2, 3, 4, 5, 7, and 8 is also provided in Tables 1 and 2.

### Example 5: Measurement of T₅₀

A 10 mg/mL solution of transaminase enzyme lyophilized cell-free lysate was prepared in 100 mM triethanolamine pH 7.5 with 0.1 mM PLP. 50 μL of the enzyme solution was aliquoted into PCR plates in triplicate for each tested temperature. The enzyme was heat challenged for 10 minutes at temperatures ranging from 35° C. to 95° C. in a thermocycler. Plates were cooled to room temperature and enzymatic reactions were assembled at a final enzyme concentration of 0.5 g/L with heated and non-heat-challenged controls, following the reaction protocol described in Example 3. The percentage of remaining enzyme activity versus temperature in ° C. were plotted to determine Tso values.

### Example 6: Measurement of Rates

Reactions were performed as described in Example 4, except with 7.5 mg/mL lyophilized transaminase cell-free powder instead of 11.25 mg/mL. Conversion was measured every 20 minutes between 0 and 80 minutes, and plotted with linear regression to obtain the rate. Enzyme properties for which improvement is desirable include, but are not limited to, enzymatic activity, thermal stability or, stereoselectivity. The improvements can relate to a single enzyme property, such as enzymatic activity, or a combination of different enzyme properties, such as enzymatic activity and stereoselectivity.

Table 1 below provides a list of the SEQ ID NOs disclosed herein with associated activities. The amino acid sequences below are based on the transaminase sequence of SEQ ID NO: 1, unless otherwise specified. In Table 1, each row lists a SEQ ID NO. The column listing the number of mutations (i.e., residue changes) refers to the number of amino acid substitutions as compared to the transaminase enzyme of SEQ ID NO: 1. In Table 1, d.r. is used to denote “diastereomeric ratio”. The first digit denotes the fraction of the product that is the (1S,4R,5R)-6,8-dioxabicyclo[3.2.1]octan-4-amine, while the second digit denotes the fraction of the product that is (1S,4S,5R)-6,8-dioxabicyclo[3.2.1]octan-4-amine. Some of the enzymes described herein display improved thermostability relative to the parent enzyme. Tso is used as a measure of the thermostability of an enzyme, and describes the temperature of half-inactivation, measured by performing a heat challenge of the enzyme for 10 minutes at a range of temperatures between 35 and 95° C. Table 2 below provides rates of reaction associated with given SEQ ID NOs. Some of the enzymes described herein display improved rate of reaction relative to the parent enzyme.

TABLE 1
Activity of Transaminase Enzymes

| | | Number of | | | |
|---|---|---|---|---|---|
| | | Changes | | | |
| SEQ | Residue Changes Relative | Relative to | | | T₅₀, |
| ID NO: | to SEQ ID NO: 1 | SEQ ID NO: 1 | Conversion | d.r. | ° C. |
| 1 | — | 0 | 50 | 18:1 | 71.1 |
| 2 | A5L | 1 | 60 | 17:1 | 71.6 |
| 3 | A5L; I55V; I122M; F215H; E256R | 5 | 63 | 25:1 | 68.4 |
| 4 | A5L; I55V; I122M; A192S; G193I; | 7 | 73 | 20:1 | 76.4 |
| | F215H; I263M; | | | | |
| 5 | A5L; I55V; V69A; I122M; A192S; | 8 | 58 | 36:1 | 76.4 |
| | G193I; F215H; I263M; | | | | |
| 6 | A5L; P48V; I55V; T62A; V69A; F88W; | 13 | 65 | 73:1 | 83.2 |
| | I122M; W124G; A192S; G193I; F215H; | | | | |
| | E256R; I263M; | | | | |
| 7 | A5L; P48V; I55V; T62A; V69A; F88W; | 15 | 74 | 44:1 | 82.2 |
| | I122M; W124G; A126V; V152I; A192S; | | | | |
| | G193I; F215H; E256R; I263M; | | | | |
| 8 | A5L; I55V; V69A; I122M; F215H; E256R; | 6 | 47 | 50:1 | 68.1 |

TABLE 2
Rates of Transaminase Enzymes

| SEQ ID NO: | Rate (mM/min) |
|---|---|
| 1 | 0.5 |
| 2 | 0.7 |
| 4 | 1.9 |
| 5 | 2.8 |
| 6 | 2.1 |

It will be appreciated that variations of the above-discussed and other features and functions, or alternatives thereof, may be desirably combined into many other different systems or applications. It will also be appreciated that various alternatives, modifications, variations or improvements therein may be subsequently made by those skilled in the art, and are also intended to be encompassed by the following claims.

What is claimed is:

1. A transaminase enzyme selected from SEQ ID NO: 2, SEQ ID NO: 3, SEQ ID NO: 4, SEQ ID NO: 5, SEQ ID NO: 6, SEQ ID NO: 7, or SEQ ID NO: 8.

2. The transaminase enzyme of claim 1 comprising an amino acid sequence having at least a 90% sequence identity to SEQ ID NO: 2.

3. A polynucleotide encoding the transaminase enzyme of claim 2.

4. The polynucleotide of claim 3, comprising SEQ ID NO: 10.

5. The transaminase enzyme of claim 1 comprising an amino acid sequence having at least a 90% sequence identity to SEQ ID NO: 3.

6. A polynucleotide encoding the transaminase enzyme of claim 5.

7. The polynucleotide of claim 6, comprising SEQ ID NO: 11.

8. The transaminase enzyme of claim 1 comprising an amino acid sequence having at least a 90% sequence identity to SEQ ID NO: 4.

9. A polynucleotide encoding the transaminase enzyme of claim 8.

10. The polynucleotide of claim 9, comprising SEQ ID NO: 12.

11. The transaminase enzyme of claim 1 comprising an amino acid sequence having at least a 90% sequence identity to SEQ ID NO: 5.

12. A polynucleotide encoding the transaminase enzyme of claim 11.

13. The polynucleotide of claim 12, comprising SEQ ID NO: 13.

14. The transaminase enzyme of claim 1 comprising an amino acid sequence having at least a 90% sequence identity to SEQ ID NO: 6.

15. A polynucleotide encoding the transaminase enzyme of claim 14.

16. The polynucleotide of claim 15, comprising SEQ ID NO: 14.

17. The transaminase enzyme of claim 1 comprising an amino acid sequence having at least a 90% sequence identity to SEQ ID NO: 7.

18. A polynucleotide encoding the transaminase enzyme of claim 17.

19. The polynucleotide of claim 18, comprising SEQ ID NO: 15.

20. The transaminase enzyme of claim 1 comprising an amino acid sequence having at least a 90% sequence identity to SEQ ID NO: 8.

21. A polynucleotide encoding the transaminase enzyme of claim 20.

22. The polynucleotide of claim 21, comprising SEQ ID NO: 16.

23. An expression vector comprising the transaminase enzymes of claim 1, operably linked to one or more control sequences suitable for directing expression of the encoded polypeptide in a host cell.

24. The expression vector of claim 23, wherein the control sequence comprises a promoter.

25. The expression vector of claim 24, wherein the promoter comprises an E. coli promoter.

26. A host cell comprising the expression vector of claim 23.

27. The host cell of claim 26, wherein said host cell is E. coli.
